(19) United States
(12) Patent Application Publication
(10) Pub. No.: US 2002/0187179 A1
(43) Pub. Date: Dec. 12, 2002

(54) Non-steroidal anabolic compositions and associated methods

(75) Inventors: Trevor Page, Salt Lake City, UT (US); Darald Donnell, Salt Lake City, UT (US)

Correspondence Address: THORPE NORTH WESTERN, 8180 SOUTH 700 EAST, SUITE 200, P.O. BOX 1219, SANDY, UT, 84070, US

(73) Assignee: Lifesmart Nutrition, Inc.

(21) Appl. No.: 09/847,530

(22) Filed: May 2, 2001

(51) Int. Cl. A61K 31/7048; A61K 31/343

(52) U.S. Cl. 424/439; 514/27; 514/456

(57) ABSTRACT

The present invention provides a non-steroidal anabolic formulation which reduces or eliminates an objectionable isoflavone taste, while simultaneously increasing the efficiency of an isoflavone dose. In one aspect, an effective amount of methoxyflavone, and optionally an effective amount of ipriflavone, are uniformly dispersed in a caramel or taffy composition having an insulin level enhancing amount of sugar. In another aspect, the caramel composition may include an effective amount of an invert sugar.

### FIELD OF THE INVENTION

The present invention relates generally to a composition and method for administering non-steroidal anabolic agents. More particularly, it concerns formulations which enhance metabolism and absorption of non-steroidal anabolic agents.

### BACKGROUND OF THE INVENTION

Modern trends in health and fitness have increasingly focused on the desirability of the development and growth of muscle tissue. In addition to the obvious benefits of significant muscle mass, such as strength, other positive health benefits, such as increased basal metabolism have been reported. To this end, a variety of dietary supplements which are administered for the purpose of promoting weight gain and muscle tissue growth have been introduced to the health and fitness market.

While anabolic muscle growth is desired, it has been recognized that many anabolic activity inducing agents, such as steroidal compounds, also produce significant undesirable side effects when administered in significant quantity. Therefore, non-steroidal anabolic activity inducing agents have been highly sought. One such agent is 5-methyl-isoflavone and derivatives thereof. U.S. Pat. No. 4,163,746 which is incorporated herein by reference in its entirety, discloses a variety of 5-methyl-isoflavone derivatives and their use as a feed supplement for various animals. Further, U.S. Pat. No. 3,864,362, which is incorporated herein by reference in its entirety, discloses a variety of anabolic activity inducing isoflavones.

In addition to anabolic activity inducing agents, other isoflavone agents have shown an ability to improve or prevent conditions that limit or decrease physical ability, such as osteoporosis. To this end, G.B. Patent 1,360,461, U.S. Pat. No. 3,833,730, and U.S. Pat. No. 4,826,963, each of which are incorporated herein by reference in their entirety, disclose various complexes and derivatives of 7-isoproproxy-isoflavone for treating and preventing osteoporosis.

Unfortunately, isoflavones as well as many other bioflavanoids have an undesirable taste. To this end, and in order to allow for convenience, many isoflavone containing supplements are produced as pills or tablets which are swallowed. However, such oral dosage formulations do not maximize isoflavone dosage efficacy, and may still result in a residual or after taste.

Other oral isoflavone formulations have included a variety of ingredients in a liquid drink or shake formulation. Such formulations generally provide one or more flavorants which attempt to mask the isoflavone taste. Unfortunately, liquid drinks and shakes are not conveniently transported and consumed throughout the day, as they require water and/or other materials for preparing the dosage for consumption.

As a result, current research and development efforts continue to seek isoflavone and other bioflavanoid dosage formulations which are easily consumable, portable, and that maximize active ingredient metabolism and absorption by the body.

### SUMMARY OF THE INVENTION

Accordingly, the present invention provides a non-steroidal anabolic formulation which includes a caramel or taffy base having an effective amount of methoxyisoflavone, and optionally ipriflavone dispersed therein. In one aspect, the amount of methoxyflavone may be from about 0.05% to about 17% w/w of the formulation. In another aspect, the amount of methoxyflavone may be about 3.5% w/w of the formulation. In yet another aspect, the amount of ipriflavone may be from about 0.05% to about 17% w/w of the formulation. In a further aspect, the amount of ipriflavone may be about 0.25% w/w of the formulation.

The amount of sugar in the caramel or taffy base of the present invention may be an amount sufficient to mask or reduce the objectionable isoflavone taste, and may also be sufficient to rapidly enhance insulin levels. The total sugar in the caramel base may include an effective amount of an invert sugar. In one aspect, the amount of invert sugar may be from about 1% to about 20% w/w of the formulation. In another aspect the amount of invert sugar may be from about 3% to 15% w/w of the formulation.

A variety of invert sugar types may be utilized with the present invention to provide a heightened sweetening effect. In one aspect, the invert sugar may be a mixture of dextrose (i.e. D-glucose) and fructose. In another aspect, the dextrose and the fructose may each be present in an amount of about 50% w/w of the invert sugar. In yet another aspect, the invert sugar may be provided by rice syrup and include a mixture of glucose and maltose. In a further aspect, the amount of rice syrup may be from about 15% to about 40% w/w of the formulation.

The present invention also encompasses a method for making a non-steroidal anabolic formulation. In describing the method the terms “caramel” and “taffy” may be used interchangeably. In one aspect, a method of making a non-steroidal anabolic formulation comprises the steps of: a) preparing a caramel base containing an effective amount of an invert sugar in a conventional manner in a heated liquid caramel phase form; b) partially cooling said heated liquid caramel phase to a temperature at which methoxyflavone is stable; c) adding a desired amount of methoxyflavone to said partially cooled liquid caramel phase; d) agitating said partially cooled liquid caramel phase until the methoxyflavone is substantially uniformly dispersed therein; and e) further cooling said partially cooled liquid caramel phase to a solid thereby resulting in said non-steroidal anabolic formulation. Such a method may further include the step of adding an effective amount of ipriflavone.

In one aspect, the temperature of said partially cooled liquid caramel phase may be between about 160° F. to about 220° F. at the time said methoxyflavone and ipriflavone are added. In another aspect, the temperature is from about 180° F. to 200° F.

In one aspect, the amount of methoxyflavone added may be from about 0.1% to about 18% w/w of the non-steroidal anabolic formulation. In another aspect, the amount of methoxyflavone may be about 4.5% w/w of the formulation.

In one aspect, the amount of ipriflavone added maybe from about 0.1% to about 18% w/w of the non-steroidal anabolic formulation. In another aspect, the amount of ipriflavone added may be about 0.5% w/w of the formulation.

Another method included in the present invention is a method of increasing the in vivo efficacy of an isoflavone dose. Such a method may include the steps of: a) distributing an effective amount of methoxyflavone into a caramel composition containing an insulin enhancing amount of sugar and an invert sugar to form an isoflavone dose; and b) orally administering the dose to a human. In one aspect, the distributing step of such a method may include the step of distributing an effective amount of ipriflavone into the caramel composition.

In one aspect, the amount of invert sugar may be from about 1% to about 20% w/w of the formulation. In another aspect, the amount of invert sugar may be about 3% to 15% w/w of the formulation. In a further aspect, the invert sugar includes a mixture of dextrose and fructose. In yet another aspect, the dextrose and the fructose may each be present in an amount of about 50% w/w of the invert sugar. In an additional aspect, the invert sugar may be provided by rice syrup and includes a mixture of glucose and maltose. In yet another aspect, the amount of rice syrup is from about 15% to about 40% w/w of the formulation.

In addition to the above-recited methods, the present invention includes a method of improving the palatability of an isoflavone extract. Such a method may include the steps of: a) distributing an effective amount of the isoflavone into a caramel composition containing a sufficient amount of invert sugar to reduce or eliminate any disagreeable taste caused by the isoflavone.

In one aspect, the amount of invert sugar may be from about 1% to about 20% w/w of the formulation. In another aspect, the amount of invert sugar may be from about 3% to 15% w/w of the formulation. In a further aspect, the invert sugar may include a mixture of dextrose and fructose. In yet another aspect, the dextrose and the fructose are each present in an amount of about 50% w/w of the invert sugar. In an additional aspect, the invert sugar may be provided by rice syrup and includes a mixture of glucose and maltose. In another aspect, the amount of rice syrup is from about 15% to about 40% w/w of the formulation.

The present invention additionally provides a method of facilitating anabolic activity in muscle cells comprising the step of administering an isoflavone dosage to a subject, wherein said isoflavone dosage includes a chew base having a therapeutically effective amount of methoxyflavone dispersed therein. Further components included in the isoflavone dosage may be as disclosed herein.

There has thus been outlined, rather broadly, the more important features of the invention so that the detailed description thereof that follows may be better understood, and so that the present contribution to the art may be better appreciated. Other features of the present invention will become clearer from the following detailed description of the invention, taken with the accompanying claims, or may be learned by the practice of the invention.

### DETAILED DESCRIPTION

Definitions

Before the present non-steroidal anabolic agent formulations are disclosed and described, it is to be understood that the present invention is not limited to the particular process steps and materials disclosed herein, but is extended to equivalents thereof as would be recognized by those ordinarily skilled in the relevant arts. It should also be understood that terminology employed herein is used for the purpose of describing particular embodiments only and is not intended to be limiting.

In describing and claiming the present invention, the following terminology will be used.

The singular forms “a,” and, “the” include plural referents unless the context clearly dictates otherwise. Thus, for example, reference to a caramel containing “a flavone” includes one or more flavone components, reference to “a sugar” includes reference to one or more sugars, and reference to “the flavorant” includes reference to one or more flavorants.

In describing and claiming the present invention, the following terminology will be used in accordance with the definitions set forth below.

As used herein, “isoflavone” refers to either methoxyflavone, ipriflavone, or a combination thereof.

As used herein, “methoxyflavone” and “methoxyisoflavone” may be used interchangeably and refer to a non-steroidal anabolic activity inducing compound having the general formula:

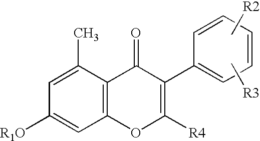

wherein R₁ is a hydrogen or substituted or unsubstituted alkyl or substituted or unsubstituted aralkyl; R₂ and R₃ are each a hydrogen, alkyl, or alkoxy group; and R₄ is a hydrogen or alkyl group. In one aspect, when R₁ is a substituted or unsubstituted alkyl, the alkyl may be a straight or branched chained alkyl group having 1-20 carbon atoms. In another aspect, the substituents such alkyl group may be a hydroxy or C₁₋₁₆ alkoxy group (i.e. methyl, ethyl, isopropyl, hydroxyethyl, 2-methyl-propyl, 3-methyl-butyl, hexadecyl, etc.). In yet another aspect, when R₁ is an optionally substituted aralkyl group, it may contain from about 7 to about 9 carbon atoms (i.e. benzyl, beta-phenyl-ethyl). In one aspect, R₂, R₃, and R₄ are each hydrogen Examples of specific compounds include without limitation, 5-methyl-7-methoxy-isoflavone, 5-methyl-alkoxy-isoflavone, 5-methyl-7-ethoxy-isoflavone, 5-methyl-7-(2-hydroxy-ethoxy)-isoflavone, 5-methyl-7-isopropoxy-isoflavone, as well as mixtures and derivatives thereof.

As used herein, “ipriflavone” refers to a compound known by various IUPAC names, including 7-(1-methylethoxy)-3-phenyl-4H-1-benzopyran-4-one; 7-isopropoxy-3-phenyl-4H-1-benzopyran-4-one; 7-isopropoxy-3-phenylchromone; and 7-isopropoxyisoflavone. Ipriflavone is listed as a calcium regulator at page 870, entry no. 5090 in The Merck Index (12^{th} ed. 1996).

The terms “formulation” and “composition” may be used interchangeably herein.

As used herein, a “sugar” refers to any type of simple carbohydrate, such as a mono or disaccharide, or a combination thereof, either naturally obtained, refined from a natural source, or artificially produced, which may act as a suitable sweetener in a caramel composition.

As used herein, “invert sugar” refers to a combination of two or more sugars, either naturally obtained, refined from a natural source, or artificially produced, that produces a greater sweetness than a single type of sugar. By way of example without limitation, an invert sugar may include a mixture of fructose and D-glucose in substantially equal parts. One example of a naturally obtained invert sugar is rice syrup. Rice syrup is generally obtained by culturing rice with certain enzymes to break down starches, straining off the liquid, and cooking the remaining portion until a desired consistency is reached. The resultant product contains a mixture of soluble complex carbohydrates, maltose, and glucose. In such a case, the combination of maltose and glucose act much like the more traditional combination of fructose and D-glucose.

As used herein, “chew” and “chew base” may be used interchangeably and refer to either a caramel or taffy base.

As used herein, “caramel,” and “caramel base,” may be used interchangeably, and refer to a smooth, chewy composition made with sugar, butter or other fats, cream or milk or milk solids, and flavoring. Such ingredients may be unaltered natural products, natural products which are processed or refined, or may be fully synthesized products.

As used herein, “taffy,” or “taffy base” refers to a chew candy or confection which is made with various types of sugars, including but not limited to simple sugars, invert sugars, brown sugars, and molasses, which is boiled until very thick and then pulled until it is glossy and holds its shape.

As used herein, “artificial sweetener” refers to a sweetening agent which does not provide a substantial amount of calories when consumed, as compared to the calories provided by an amount of sugar required to impart an equivalent sweetening effect. A variety of artificial sweeteners are known to those skilled in the art, including without limitation, saccharin, aspartame, sucralose, etc.

As used herein, an “effective amount,” and “sufficient amount” may be used interchangeably and refer to an amount of an ingredient which, when included in a chew composition, is sufficient to achieve an intended compositional or physiological effect. For example, a “sufficient amount” of invert sugar would be the minimum amount needed to reduce or eliminate a disagreeable taste caused by an amount of isoflavone. Further, a “therapeutically effective amount” refers to an amount of methoxyflavone or ipriflavone which is sufficient to achieve a desired physiological effect. The determination of an effective amount is well within the ordinary skill in the art of pharmaceutical, neutraceutical, herbaceutical, cosmetic, and medical sciences. See, for example, Meiner and Tonascia, “Clinical Trials: Design, Conduct, and Analysis,” Monographs in Epidemiology and Biostatistics, Vol. 8 (1986), incorporated by reference in its entirety.

As used herein, “an insulin enhancing amount,” or “an insulin level enhancing amount” of a substance refers to an amount of sugar or other nutritional agent that is sufficient to produce or raise the amount of insulin in the blood to a level which increases the metabolism and absorption of methoxyflavone or ipriflavone. Various methods for measuring and determining various insulin levels and their effect on the metabolism and absorption of various nutritional components are well known to those in the art.

As used herein, “active agent” refers to an agent such as methoxyflavone or ipriflavone which imparts, or is capable of imparting or inducing an intended measurable physiological effect when administered to the body. Examples of additional active agents include vitamins, minerals, other herbal and medicinal compounds, etc.

Concentrations, amounts, and other numerical data may be expressed or presented herein in a range format. It is to be understood that such a range format is used merely for convenience and brevity and thus should be interpreted flexibly to include not only the numerical values explicitly recited as the limits of the range, but also to include all the individual numerical values or sub-ranges encompassed within that range as if each numerical value and sub-range is explicitly recited.

As an illustration, a concentration range of “about 0.1% w/w to about 17% w/w” should be interpreted to include not only the explicitly recited concentration of about 0.1% to about 17% w/w, but also include individual concentrations and the sub-ranges within the indicated range. Thus, included in this numerical range are individual concentrations such as 2% w/w, 5% w/w, and 6% w/w, and sub-ranges such as from 1% w/w to 3% w/w, from 2% w/w to 6% w/w, from 8% w/w to 16% w/w, from 5% w/w to 10% w/w, etc. The same principle applies to ranges reciting only one numerical value.

Similarly, a range recited as “less than about 3.8% w/w” should be interpreted to include all of the values and ranges as elaborated above for the range of “from about 0.1% w/w to about 25% w/w.” Furthermore, such an interpretation should apply regardless of the breadth of the range or the characteristics being described.

Invention

The present invention is drawn to an oral dosage methoxyflavone formulation which includes a therapeutically effective amount of methoxyflavone, and optionally a therapeutically effective amount of ipriflavone contained in a chewy confection type vehicle such as a caramel or taffy. Such a formulation provides significantly improved taste, convenience, and efficacy aspects over conventional liquid, shake, tablet, or capsule formulations.

Single methoxyflavone formulation dosage sizes typically range from about 1 to about 10 grams total weight and are individually wrapped for convenient transport and administration. The amount of methoxyflavone contained in a chew of this size may be from about 0.5 mg to about 1 g, and the amount of ipriflavone may be from about 0.5 mg to about 1 g when present. In one aspect, the total weight for a single dosage may be about 5.8 g, and the amount of methoxyflavone contained therein may be about 200 mg. Further, the amount of ipriflavone included in a chew of this size may be about 15 mg, when included.

The methoxyisoflavone and ipriflavone components of the present invention may be included as either a powder or a liquid. Further, both of these components may be obtained by a variety of process including extraction and synthesis which are known to those skilled in the art.

The amount of methoxyisoflavone component contained in the formulation may be varied by one skilled in the art without undue experimentation in order to achieve a particularly desired result. However, the methoxyisoflavone content may generally be from about 0.05% w/w to about 17% w/w of the formulation. In one aspect, the amount may be from about 0.1% w/w to about 7% w/w of the formulation. In another aspect, the amount may be about 3.5% w/w of the formulation.

The amount of ipriflavone component contained in the formulation of the present invention, when included, may also be varied according to the knowledge of one skilled in the art in order to achieve a particularly desired result. However, the ipriflavone content may generally be from about 0% w/w to about 17% w/w of the formulation. In one aspect, it may be from about 0.05% w/w to about 5% w/w of the formulation. In another aspect, it may be about 0.25% w/w of the formulation.

Invert sugar has found to be particularly good at combating the disagreeable taste of isoflavones is due to its particular nature. Invert sugar is generally a combination of the simple sugars dextrose (D-glucose) and fructose which provides a level of sweetness exceeding that of a single type of sugar. In one aspect, the invert sugar may be a product of the action of the enzyme invertase on sucrose to form a mixture of levulose fructose) and D-glucose (dextrose). However, invert sugar, as defined herein, may be any combination of simple sugars which imparts a heightened sweetness. In one aspect, the invert sugar used may be that containing an equal parts mixture of D-glucose and fructose. In another aspect, the invert sugar used may be a combination of maltose and glucose as provided by, for example, rice syrup.

Each of the invert sugar components works in a complimentary, or synergistic manner in order to provide superior, or heightened sweetness. Particularly, such synergy is thought to come from the time variation of interaction between the various sugars and the taste buds. Particularly, the glucose, and maltose or fructose, as simple sugars, provide an initial burst of sweetness as the invert sugar enters the mouth. This quick sweetening effect masks the initial distaste of the isoflavones. The sucrose and corn syrup solids used in making the chew base, being either a disaccharide or starch hydrolysis product, provide a sustained sweetening power during chewing. Further, the maltose or fructose, while involved in the above two mentioned states, are also believed to provide a lingering sweetness which masks the objectionable isoflavone aftertaste or residual taste.

Notably, a variety of artificial sweeteners may also be used to mask the objectionable isoflavone taste. In one aspect, one or more artificial sweeteners may be used in addition to the sugars present in the chew formulation. In another aspect, one or more artificial sweeteners may take the place of one or more of the sugars present in the chew formulation.

In addition to improving the taste, or palatability, and convenience of an isoflavone dose, the chew vehicle of the present oral delivery formulation also improves the overall dosage efficacy. Particularly, methoxyflavone and ipriflavone are known to be insulin sensitive agents. That is to say that the absorption of these agents into the cells for metabolism and use in imparting an anabolic effect is enhanced by the presence of insulin. Insulin is generally known to those skilled in the art as a gate keeping substance which in part regulates the transport of various agents, particularly nutrients and other agents involved in cellular metabolism.

As such, the chew vehicle of the present invention is a particularly well suited vehicle for administering isoflavones between meals, or at any other time when blood insulin levels may be at a minimum, due to the total amount of combined sugars which are present. Particularly, the total amount of sugars in a single chew dosage is sufficient to raise insulin levels to a point which enhance isoflavone metabolism and absorption by the cells.

The caramel or taffy composition of the present invention may be a preparation of any combination of ingredients which is known to those ordinarily skilled in the art of making caramel, taffy, or other confections, and is not limited except by a requirement to contain an effective amount of methoxyflavone and/or ipriflavone.

In addition to the methoxyflavone and/or ipriflavone active ingredients, other active ingredients may be included in the formulation of the present invention which impart a positive health benefit. As will be recognized by those skilled in the art, a wide variety of positive health benefit imparting ingredients may be selected from herbal and botanical extracts, as well as medicinal compounds and be added as desired in order to achieve a specific therapeutic result, or to further enhance the anabolic process. Such additions may be made by the skilled artesian without undue experimentation. In one aspect, the amount of any one specific positive health benefit imparting substance may be from about 0% to about 20%, or any amount in between, as determined by one skilled in the art to achieve a desired formulation result.

Generally, herbal and botanical extracts are made from all kinds of herb and botanic sources and formulated based on their therapeutic function. For example positive health benefits such as, bone/joint, brain function, cardiovascular, circulatory, diet, depression, digestion, energy, eye vision, general health, immune system, liver, men's health respiratory, rest, urinary tract, women's health, etc., may be derived when sufficient quantities of herbal or botanical extracts are used. In one aspect, herbal and botanical extracts for inclusion in the present formulation can be selected from, but not limited to, Ginseng, Ginko Biloba, Dong Quai, Hawthorn berry, St. John's Wort, Saw Palmetto, Kava Kava, Rose Hips, Echinacea, Licorice Root, Grape seed, Chammomile, Sea Buckthorn, Aloe Vera, Cinnamon Bark, Cordyceps, Ho Shou Wu, Dandelion, Gynostemma, mushroom, Notginseng, Dan Shen, and mixtures thereof may be included.

In one aspect, vitamins either water soluble or oil soluble may be added. Water soluble vitamins specifically contemplated by the present invention include, but are not limited to: vitamin B₁, B₂, B₃, B₅, B₆, B₁₂, B₁₃, B₁₅, B₁₇, biotin, choline, folic acid, inositol, para-aminobenzoic acid (PABA), vitamin C, and vitamin P. Additionally, oil soluble vitamins include, but are not limited to: vitamin A, vitamin D, vitamin E, and vitamin K.

Other health imparting substances which may be combined with the desired methoxyflavone and/or ipriflavone active ingredients in the formulation of the present invention include amino acids, ionic minerals, and naturally occurring anti-oxidants. The amino acids contemplated include: alanine, arginine, carnitine, gamma-aminobutyric acid (GABA), glutamine, glycine, histidine, lysine, methionine, N-acetyl cysteine, ornithine, phenylalanine, taurine, tyrosine, and valine, but are not limited thereto. Additionally, the ionic minerals contemplated by the present invention for inclusion in an embodiment of the formulation include both anions and cations. Finally, the naturally occurring anti-oxidants contemplated for the formulation of the present invention include: grape seed, beta-carotene, and co-enzyme Q-10, but are not limited thereto.

Other medicinal compounds which are routinely used in sports medicine may be included in the chew of the present formulation, such as analgesics, anti-inflammatories, agents which support good joint health, etc. Examples of such agents include without limitation, aspirin, ibuprofen, ketoprofen, acetaminophen, chondroitin, methylsulfonyl methane, glucosamine, as well as mixtures thereof.

Additionally, a variety of other muscle building components may be included in the formulation of the present invention. By way of example, without limitation, proteins and other amino acid complexes, which are well known to those skilled in the art may be utilized in combination with the isoflavones in order to provide enhanced muscle growth.

In one aspect, the amount of invert sugar contained in the prepared chew composition of the present invention may be from about 1% to about 20% w/w of the chew composition. In another aspect, the amount of invert sugar may be from about 3% w/w to 15% w/w of the chew composition. In yet another aspect, the amount of invert sugar may be from about 5% w/w to about 10% w/w of the chew composition. These amounts of invert sugar are in addition to the amount of table sugar (sucrose) or corn syrup solids required by the particular caramel or taffy recipe employed.

As defined above, a basic caramel formulation also contains butter or other fats, and either cream, milk, or milk products. Further, a basic taffy formulation may also contain molasses. The exact types and amounts of each of these ingredients may vary depending on the desired characteristics of the final product. Such exact amounts and types may be readily determined by one ordinarily skilled in the art.

Other ingredients known to the applicant as useful for making an isoflavone containing chew include but are not limited to: water, corn syrup, one or more oils, such as hydrogenated soybean oil, mono and diglycerides, emulsifiers, lecithin, whey solids, sweetened condensed milk, and flavorants, such as vanillin and chocolate.

The corn syrup may be selected from a variety of corn syrup types, and may be added in an amount required to achieve a specific result as determined by one skilled in the art. However, in one aspect, the corn syrup may be included in an amount of from about 18% w/w to about 40% w/w. In another aspect, the amount may be about 28% w/w.

The water included in the chew base of the present invention may be either distilled or untreated tap water, and may be added in an amount determined by one skilled in the art according to a specific recipe in order to achieve a specific result. Further, the difference between the water added and the water in the final composition due to processing effects are explained in greater detail below. However, in one aspect, the amount of water may be from about 10% to about 30%. In another aspect, the amount of water may be about 20%.

The sweetened and condensed milk may be either skim or whole as desired to impart a specific texture, and may be provided in a powdered form. In one aspect, the amount of sweetened and condensed milk may be from about 13% w/w to about 18% w/w of the formulation. In another aspect, the amount may be about 14% w/w.

In one aspect, the amount of whey may be from about 10% w/w to about 17% w/w of the formulation. In another aspect, the amount may be about 13% w/w.

In one aspect, the amount of mono and diglycerides may be from about 0.25% w/w to about 2% w/w of the formulation. In another aspect, the amount may be about 0.5% w/w.

In one aspect, the amount of lecithin may be from about 0.05% w/w to about 0.2% w/w of the formulation. In another aspect, the amount may be about 0.1% w/w.

In one aspect the amount of chocolate flavor may be from about 4% w/w to about 8% w/w of the formulation. In another aspect, the amount may be about 6% w/w.

The flavorant may be selected from a variety of flavorants known to those skilled in the art. In one aspect, the flavorant may be vanillin. In another aspect, the flavorant may be chocolate liquor. In another aspect, the amount of flavorant may be from about 0.1% w/w to about 0.4% w/w of the formulation. In another aspect, the amount may be about 0.2% w/w.

In one aspect, the chew base, may be partially or entirely made utilizing natural ingredients. Natural invert sugar sources such as rice syrup and sugar sources such as evaporated cane juice (turbinado sugar) may be used as one or more sweetening ingredients. Coconut oil and mono and diglycerides from vegetable or other natural sources may be used as oil and fat ingredients. Further ingredients which may be used include without limitation Soya lecithin, and natural flavorings, including chocolate and vanilla.

Those of ordinary skill in the art will recognize that the amount of each of the above-recited natural ingredients may be varied in order to achieve a particularly desired result. However, in one aspect, the amount of rice syrup may be from about 10% w/w to about 40% w/w of the formulation. In another aspect, the amount of rice syrup may be about 36% w/w of the formulation. Of particular note is that in general, rice syrup is approximately 48% maltose and glucose and 52% complex carbohydrates. As such, the range of effective invert sugar component provided by the rice syrup may be from about 5% w/w to about 20% w/w.

In one aspect, the amount of turbinado sugar may be from about 15% w/w to about 20% w/w of the formulation. In another aspect, the amount may be about 18% w/w of the formulation.

In one aspect, the amount of coconut oil may be from about 1% w/w to about 5% w/w of the formulation. In another aspect, the amount may be about 2%.

In one aspect, the amount of Soya lecithin may be from about 0.05% w/w to about 0.2% w/w of the formulation. In another aspect, the amount may be about 0.1% w/w.

In one aspect, the amount of mono and diglycerides from vegetable sources may be from about 0.25% w/w to about 2% w/w of the formulation. In another aspect, the amount may be about 0.5% w/w.

When a taffy base is used, any of the above recited sugars may be used, in addition to an amount of molasses which is sufficient for making taffy. Those skilled in the art will readily recognize the proper range of molasses sources and amounts which may be used in order to formulate a taffy base having specifically desired characteristics.

A method for making the isoflavone formulation of the present invention encompasses all methods for making caramel or taffy which are known to those ordinarily skilled in the art thereof. Generally, the isoflavone ingredients may be added at any time during the process. However, in one aspect to ensure maximal isoflavone distribution, and minimize isoflavone loss by changing forms, decomposition, etc, the timing of isoflavone addition and temperature to which it is subjected may be regulated.

Therefore, in a one aspect of the invention, the isoflavone ingredients are added to the chew composition, after the heated composition has been cooled to a temperature at which methoxyflavone and ipriflavone will not degrade. In another aspect, in order to achieve uniform distribution, and ensure isoflavone stability, the temperature will be from about 140° F. to about 230° F., and most preferably, the temperature will be about 160° F. to about 210° F.

In order to achieve uniform distribution of the methoxyflavone and the ipriflavone, the chew composition may be sufficiently agitated after adding them. In one aspect, the chew composition is continuously cooled and agitated after the addition of the isoflavones until the composition is sufficiently solid that agitation is not practical. At this point the chew is ready to be divided into individual pieces for packaging. When a taffy base is used, the composition may be pulled after cooling until the desired consistency is reached, prior to division for individual packaging.

Because of the heating and stirring process under which most caramel or taffy compositions are prepared, the amount of ingredient added during processing will vary somewhat from the amount retained in the finally prepared chew composition. This is mostly due to the evaporation of water out of the various components which yields a final composition having a greater percentage of some ingredients which are unaffected by the removal of water. Therefore, in order to achieve a desired methoxyflavone and/or ipriflavone amount in the prepared chew composition as enumerated above, methoxyflavone is added during processing in an amount of about 0.1% to about 18% w/w of the chew composition. In one aspect, the methoxyflavone is added during processing in an amount of about 4% to about 14% of the chew formulation. In another aspect, the methoxyflavone extract is added during processing in an amount of about 5% w/w of the chew formulation. Further, the ipriflavone, when included, is added in an amount of from about 0.1% w/w to about 18% w/w of the chew formulation. In another aspect, the ipriflavone is added in an amount of about 0.5% w/w of the formulation.

Additionally, in order to achieve the desired amount of invert sugar enumerated above, invert sugar is added during processing in an amount of about 1% to about 9% w/w of the chew composition. In one aspect, the invert sugar amount added during processing is about 5% w/w of the caramel about 8 lbs. water, and then 2.25 lbs. of invert sugar (levulose and D-glucose), and 6.16 lbs. of sweetened condensed whole milk were added to the whey and water to form a milk mixture. The milk mixture was added to the pot and heating continued until the combined mixture reached a temperature of approximately 235° F.

The mixture contained in the pot was cooled to 232° F. while stirring continued, and 1.60 lbs. of chocolate liquor was added. Mixing was continued, and the composition temperature was allowed to cool to about 200° F. Upon reaching the temperature of about 200° F., 2.0 lbs. of methoxyflavone, and 0.13 lbs. of ipriflavone were added to the caramel composition. Mixing was continued, and the composition allowed to cool to a temperature of about 180° F.

Once a temperature of about 180° F. was reached, the caramel composition was removed from the pot and transferred to a cooling table. The composition was allowed to rest upon the cooling table until it reached a temperature of about 91° F., at which time the composition was cut and wrapped into individual pieces.

The above described process yielded an isoflavone containing caramel composition having the following components in the amounts specified:

| | % Amount of | | % Amount of |
|---|---|---|---|
| Ingredient | Composition | Ingredient | Composition |
| Water | 10 | Whey Solids | 6.93 |
| Sucrose | 12.76 | Sweetened | 10.78 |
| | | Condensed | |
| | | Whole Milk | |
| Corn Syrup | 29.76 | Chocolate | 3.42 |
| | | Flavor | |
| Hydrogenated | 10.78 | Methoxy- | 3.5 |
| Soybean Oil | | flavone | |
| Emulsifier | 0.20 | Ipriflavone | 0.25 |
| Lecithin | 0.20 | | |
| Invert Sugar | 4.04 | | |

The isoflavone formulation having the components enumerated above showed excellent flavor, texture, and dissolution qualities in the mouth.

It is to be understood that the above-described arrangements are only illustrative of the application of the principles of the present invention. Numerous modifications and alternative arrangements may be devised by those skilled in the art without departing from the spirit and scope of the present invention and the appended claims are intended to cover such modifications and arrangements. Thus, while the present invention has been described above with particularity and detail in connection with what is presently deemed to be the most practical and preferred embodiments of the invention, it will be apparent to those of ordinary skill in the art that numerous modifications, including, but not limited to, variations in size, materials, shape, form, function and manner of operation, assembly and use may be made without departing from the principles and concepts set forth herein.

What is claimed is:

1. A non-steroidal anabolic formulation comprising a chew base having a therapeutically effective amount of methoxyflavone dispersed therein.

2. The non-steroidal anabolic formulation of claim 1, wherein the amount of methoxyflavone is from about 0.05% to about 17% w/w of the formulation.

3. The non-steroidal anabolic formulation of claim 2, wherein the amount of methoxyflavone is about 3.5% w/w of the formulation.

4. The non-steroidal anabolic formulation of claim 1, further comprising a therapeutically effective amount of ipriflavone.

5. The non-steroidal anabolic formulation of claim 4, wherein the amount of ipriflavone is from about 0.05% to about 17% w/w of the formulation.

6. The non-steroidal anabolic formulation of claim 5, wherein the amount of ipriflavone is about 0.25% w/w of the formulation.

7. The non-steroidal anabolic formulation of claim 1, wherein the chew base includes an effective amount of an invert sugar.

8. The non-steroidal anabolic formulation of claim 7, wherein the amount of invert sugar is from about 1% to about 20% w/w of the formulation.

9. The non-steroidal anabolic formulation of claim 8, wherein the amount of invert sugar is about 3% to 15% w/w of the formulation.

10. The non-steroidal anabolic formulation of claim 9, wherein the invert sugar comprises a mixture of dextrose and fructose.

11. The non-steroidal anabolic formulation of claim 10, wherein the dextrose and the fructose are each present in an amount of about 50% w/w of the invert sugar.

12. The non-steroidal anabolic formulation of claim 7, wherein the invert sugar comprises rice syrup and includes a mixture of glucose and maltose.

13. The non-steroidal anabolic formulation of claim 12, wherein the amount of rice syrup is from about 15% to about 40% w/w of the formulation.

14. A method of making a non-steroidal anabolic extract formulation comprising the steps of:
a) preparing a chew base containing an effective amount of an invert sugar in a conventional manner in a heated liquid phase form;
b) partially cooling said heated liquid phase to a temperature at which methoxyflavone is stable;
c) adding a desired amount of methoxyflavone to said partially cooled liquid phase;
d) agitating said partially cooled liquid phase until the methoxyflavone is substantially uniformly dispersed therein; and
e) further cooling said partially cooled liquid phase to a solid thereby resulting in said non-steroidal anabolic formulation.

15. The method of claim 14, wherein the temperature of said partially cooled liquid caramel phase is between about 140° F. to about 230° F. at the time said methoxyflavone is added.

16. The method of claim 15, wherein the temperature is from about 160° F. to 210° F.

17. The method of claim 14, wherein the amount of methoxyflavone is from about 0.1% to 18% w/w of the formulation.

18. The method of claim 17, wherein the amount of methoxyflavone is about 4.5% w/w of the formulation.

19. The method of claim 14, wherein the invert sugar is provided by an effective amount of rice syrup.

20. The method of claim 14, further comprising the step of:
adding an effective amount of ipriflavone prior to performing the step of agitating.

21. The method of claim 20, wherein the amount of ipriflavone is from about 0.1% w/w to about 18% w/w of the formulation.

22. The method of claim 21, wherein the amount of ipriflavone is about 0.5% w/w of the formulation.

23. A method of increasing in vivo isoflavone dose efficacy, comprising the steps of:
a) distributing an effective amount of an isoflavone into a chew composition containing an insulin level enhancing amount of sugar and an invert sugar to form an isoflavone dose; and
b) orally administering the isoflavone dose to a human.

24. The method of claim 23, wherein the step of distributing further comprises the step of distributing an effective amount of ipriflavone into the chew composition.

25. The method of claim 23, wherein the amount of invert sugar is from about 1% to about 20% w/w of the formulation.

26. The method of claim 25, wherein the amount of invert sugar is about 3% to 15% w/w of the formulation.

27. The method of claim 24, wherein the invert sugar comprises a mixture of dextrose and fructose.

28. The method of claim 27, wherein the dextrose and the fructose are each present in an amount of about 50% w/w of the invert sugar.

29. The method of claim 24, wherein the invert sugar is provided by rice syrup and includes a mixture of glucose and maltose.

30. The method of claim 29, wherein the amount of rice syrup is from about 15% to about 40% w/w of the formulation.

31. The method of claim 24, wherein the step of administering the composition is performed at a time when digestive juices in the upper gastrointestinal tract of a human are at a minimum.

32. A method of improving the palatability of an isoflavone extract containing chew composition comprising the step of:
a) distributing the isoflavone into a chew composition containing a sufficient amount of invert sugar to reduce or eliminate any objectionable taste caused by the isoflavone.

33. The method of claim 32, wherein the amount of invert sugar is from about 1% to about 20% w/w of the formulation.

34. The method of claim 33, wherein the amount of invert sugar is about 3% to 15% w/w of the formulation.

35. The method of claim 32, wherein the invert sugar comprises a mixture of dextrose and fructose.

36. The method of claim 35, wherein the dextrose and the fructose are each present in an amount of about 50% w/w of the invert sugar.

37. The method of claim 32, wherein the invert sugar is provided by rice syrup and includes a mixture of glucose and maltose.

38. The method of claim 37, wherein the amount of rice syrup is from about 15% to about 40% w/w of the formulation.

39. A method of facilitating anabolic activity in muscle cells comprising the step of:
administering an isoflavone dosage to a subject, said isoflavone dosage including a chew base having a therapeutically effective amount of methoxyflavone dispersed therein.

40. The method of claim 39, wherein the amount of methoxyflavone is from about 0.05% to about 17% w/w of the formulation.

41. The method of claim 40, wherein the amount of methoxyflavone is about 3.5% w/w of the formulation.

42. The method of claim 39, further comprising a therapeutically effective amount of ipriflavone.

43. The method of claim 42, wherein the amount of ipriflavone is from about 0.05% to about 17% w/w of the formulation.

44. The method of claim 43, wherein the amount of ipriflavone is about 0.25% w/w of the formulation.

45. The method of claim 39, wherein the chew base includes an effective amount of an invert sugar.

46. The method of claim 45, wherein the amount of invert sugar is from about 1% to about 20% w/w of the formulation.

47. The method of claim 46, wherein the amount of invert sugar is about 3% to 15% w/w of the formulation.

48. The method of claim 46, wherein the invert sugar comprises a mixture of dextrose and fructose.

49. The method of claim 48, wherein the dextrose and the fructose are each present in an amount of about 50% w/w of the invert sugar.

50. The method of claim 45, wherein the invert sugar comprises rice syrup and includes a mixture of glucose and maltose.

51. The method of claim 50, wherein the amount of rice syrup is from about 15% to about 40% w/w of the formulation.
